(19) United States
(12) Patent Application Publication
(10) Pub. No.: US 2018/0211727 A1
(43) Pub. Date: Jul. 26, 2018

(54) Automated Evidence Based Identification of Medical Conditions and Evaluation of Health and Financial Benefits Of Health Management Intervention Programs

(71) Applicant: BaseHealth, Inc., Sunnyvale, CA (US)

(72) Inventors: Hadi Zarkoob, Sunnyvale, CA (US); Harshna Kapashi, Sunnyvale, CA (US); Prakash Menon, Sunnyvale, CA (US); Jason Pyle, Sunnyvale, CA (US); Emin Martinian, Sunnyvale, CA (US); Hossein Fakhrai-Rad, Sunnyvale, CA (US)

(73) Assignee: BaseHealth, Inc., Sunnyvale, CA (US)

(21) Appl. No.: 15/878,260

(22) Filed: Jan. 23, 2018

Related U.S. Application Data
(60) Provisional application No. 62/478,522, filed on Mar. 29, 2017.
(60) Provisional application No. 62/450,002, filed on Jan. 24, 2017.

(51) Int. Cl. G16H 50/30; G06F 19/00; G16H 10/60; G06N 99/00

(52) U.S. Cl. CPC G16H 50/30; G06N 99/005; G16H 10/60; G06F 19/707

(57) ABSTRACT

Certain embodiments of the present invention relate generally to using machine learning or other automated techniques to among other things, identify, estimate, and/or predict patient health conditions. Furthermore, certain embodiments of the present invention are related to health interventions performed to reduce the risk of developing diseases and health conditions. This risk reduction improves the overall health of the individual and/or the population and helps reduce healthcare costs.

### CROSS-REFERENCE TO RELATED APPLICATION

This application is related to and claims the benefit and priority of U.S. Provisional Patent Application No. 62/478,522, filed Mar. 29, 2017, the entirety of which is hereby incorporated herein by reference. This application is also related to and claims the benefit and priority of U.S. Provisional Patent Application No. 62/450,002, filed Jan. 24, 2017, the entirety of which is hereby incorporated herein by reference.

### BACKGROUND

### Field

Certain embodiments of the present invention relate generally to using machine learning or other automated techniques to among other things, identify, estimate, and/or predict patient health conditions. Furthermore, certain embodiments of the present invention are related to health interventions performed to reduce the risk of developing diseases and health conditions. This risk reduction improves the overall health of the individual and/or the population and helps reduce healthcare costs.

### Description of the Related Art

A core goal of health care is to take information about a patient and make good decisions. Such decisions could include how to treat a patient, whether to order more tests, how to choose which preventive measures would be most beneficial, as well as how to evaluate the potential future costs and liabilities of a patient's health condition.

In the past, medical professionals tended to make most if not all such decisions. With the increasing amount of information available and the demands on the scarce time of medical professionals, there may be a growing desire to apply automated decision support tools. Decision support can include automated screening of patients, tools to help physicians make more accurate diagnosis, tools to detect fraud, and tools to manage financial risks and liabilities related to caring for large groups of patients.

Automated decision support can take a dataset of patient information records and use artificial intelligence, machine learning, or similar statistical tools and “learn” a relationship between some features of the patient data and resulting conditions of interest. This is illustrated in FIG. 1.

FIG. 1 is a block diagram representing a generic decision support system for patient data. As shown in FIG. 1, patient data 110 and a cost function 120 can serve as inputs to provide system training 130. The system training 130 can train a decision support system 140 so that it can learn relationships between the patient data and resulting conditions of interest.

Automated learning systems of this kind may be valuable for a variety of reasons. First, they can adapt to the features of a given patient population. For example, patients in the northeast region of a country may have different characteristics than those in the southwest (for example, due to environment, culture, weather, economy, and the like). Automated decision support systems can examine such data and adjust their recommendations appropriately. Second, automated systems can sometimes be applied in situations where consulting a physician could be too costly. Third, automated systems can adjust their decisions to target particular costs of different kinds of mistakes. This is achieved by customizing the cost function (See FIG. 1). Finally, automated systems have the potential to integrate large amounts of data from genetic markers, behaviors (for example, the amount of physical exercise), family history, vital statistics (for example, blood pressure), and so on which may be harder for a human to weigh optimally in a decision.

While in principle more information is better, it can make the task of processing such information more complex. For example, US Patent Application Publication No. 20120053425 states: “ . . . patient care becomes increasingly difficult when multiple variables are involved. In particular, there lacks a system and method to effect a multi-dimensional analysis.”

This is sometimes referred to as the “curse of dimensionality.” That is, as more dimensions of information are available, the complexity of using machine learning, artificial intelligence, or other statistical techniques to make sense of the data grows exponentially. This increased complexity often makes it infeasible to build automated decision support systems.

This complexity is one of the prime reasons that while many powerful statistical techniques exist in theory (for example, support vector machines, decision trees, deep learning, neural networks, and the like), it is hard to apply them in practical health care settings. In practice, most learning systems attempt to deal with the curse of dimensionality in one of two ways (both of which are suboptimal).

Some systems try to use all the relevant data, as shown in FIG. 2, but suffer poor performance because incorporating and analyzing so much data is too complex. FIG. 2 is a block diagram of a decision support system which tries to apply machine learning without reducing the dimensionality of the input data and hence ends up being suboptimal.

As shown in FIG. 2, patient data 110a and a cost function 120a can serve as inputs to provide system training 130a. However, in this case patient data 110a may be too complex, including information on genetic data, billing codes, vital statistics, patient behaviors, ethnicity, gender, and family history. In short, this may be a full data set. Thus, the system training 130a may be provided by a suboptimal learning system with too much data input. The result can be a decision support system 140a that is a poor decision support system.

Other systems extract only the most relevant data (for example, blood pressure and how much the patient exercises) while ignoring clearly relevant data (for example, the patient's lipid levels). Systems with too little data reduce the complexity to manageable levels but end up being suboptimal because they ignore relevant data, as shown in FIG. 3.

FIG. 3 is a block diagram of a decision support system which extracts a subset of the input data to use in machine learning but ends up being suboptimal due to potentially ignoring useful inputs. As shown in FIG. 3, patient data 110b is extracted at 112b from a full dataset, to provide blood pressure and lack of exercise data. This blood pressure and lack of exercise data, together with a cost function 120b, can serve as inputs to provide system training 130b. Thus, the system training 130b may be provided by a suboptimal learning system with too little data input. The result can be a decision support system 140b that is a poor decision support system.

Although being able to train an automated decision system with a custom cost function has many advantages, the difficulties illustrated in FIG. 2 and FIG. 3 often prove significant. In practice, the scientific community partially deals with the curse of dimensionality by using well trained scientists with decades of experience to conduct highly controlled, large scale, scientific studies to sort out what factors predict which diseases.

In principle, one could use the existing scientific literature to use all available data to make an evidence-based prediction of disease risks. For example, one could search through the scientific literature to find the consensus on how high blood pressure affects the risk of a heart attack. One could then do the same for how lack of exercise affects the risk of diabetes, and so on. This is no small undertaking in itself but it does at least address some aspects of the curse of dimensionality by incorporating a potentially large array of risk factors in disease risk prediction.

FIG. 4 is a block diagram of a decision support system using purely evidence based predictions and therefore unable to adapt to a custom cost function or risk factors not in the scientific literature or of special interest for a particular cohort. As shown in FIG. 4, patient data 110c is extracted at 112c from a full dataset, to provide fills prescriptions and related disease data. This fills prescriptions and related disease data serves as input to provide system training 130c. Cost function 120c cannot be used because it is a custom cost not used in the literature. The system training 130c may have no peer reviewed scientific literature, and so its factors may be ignored. Instead, scientific literature may be used to make evidence based predictions 150c with the data from the full dataset. The result can be a decision support system 140c that is a suboptimal decision support system.

More particularly, as illustrated in FIG. 4, there are at least two difficulties with an evidence based approach from the scientific literature. First, this approach does not address the issue of cost functions. The scientific literature generally tries to determine the link between a risk factor, such as high blood pressure, and a disease, such as stroke, in terms of a simple probability estimate. If there is an asymmetric cost of misdiagnosis, then this may not be the best approach. A custom cost function could more accurately capture the cost of, for example, not applying an early intervention to prevent stroke to a patient who suffers a stroke as opposed to applying the early intervention to a patient who never has a stroke.

Second, while the scientific literature is rigorous, it does not capture many potential risk factors which could be relevant. For example, whether or not a patient fills prescriptions may be highly relevant to whether that patient will have higher risk of developing a disease. This may not have been studied yet in the scientific literature, because such data is readily available to a hospital but not to researchers. Similarly, co-morbidity between diseases, such as diabetes and heart disease, may be relevant but harder to address in a scientific study due to confounding factors. In controlled settings with a wider range of data available, however, one might want to use such factors in decision support. Also, the scientific literature often requires a higher standard of proof, whereas non-medical applications, such as fraud detection, may still be interesting with less definitive evidence.

In addition to the problem of complexity, many existing machine learning techniques tend to produce complicated mappings from patient data to suggested decisions. Sometimes these complicated mappings are referred to as “black box” systems because it becomes difficult to interpret how the system maps patient data to a decision. When stakeholders cannot understand the reasoning behind a suggested decision, they are less likely to follow the suggestion.

### SUMMARY

According to certain embodiments, a method can include providing, to a learning system, a subset of data from a full dataset of patient information, wherein the subset is expected to relate to a health condition. The method can also include providing to the learning system evidence based predictions as to the health condition based on the full dataset informed by scientific literature. The method can further include providing a cost function regarding the health condition to the learning system. The method can additionally include applying the learning system to the provided subset, the evidence-based predictions, and the cost function, to provide a likelihood of the health condition.

In certain embodiments, a method can include selecting a set of risk factors for a disease for a person. The method can also include determining a total effect size and disease risk for the disease based on effect sizes of the set of risk factors. The method can further include determining an expected effect of an intervention program on the disease risk. The method can additionally include conditionally implementing the intervention program for the person based on the expected effect of the intervention program.

An apparatus, according to certain embodiments, can include at least one processor and at least one memory including computer program code. The at least one memory and the computer program code are configured to, with the at least one processor, cause the apparatus at least to provide, to a learning system, a subset of data from a full dataset of patient information, wherein the subset is expected to relate to a health condition. The at least one memory and the computer program code are also configured to, with the at least one processor, cause the apparatus at least to provide to the learning system evidence based predictions as to the health condition based on the full dataset informed by scientific literature. The at least one memory and the computer program code are further configured to, with the at least one processor, cause the apparatus at least to provide a cost function regarding the health condition to the learning system. The at least one memory and the computer program code are additionally configured to, with the at least one processor, cause the apparatus at least to apply the learning system to the provided subset, the evidence-based predictions, and the cost function, to provide a likelihood of the health condition.

An apparatus, in certain embodiments, can include at least one processor and at least one memory including computer program code. The at least one memory and the computer program code are configured to, with the at least one processor, cause the apparatus at least to select a set of risk factors for a disease for a person. The at least one memory and the computer program code are also configured to, with the at least one processor, cause the apparatus at least to determine a total effect size and disease risk for the disease based on effect sizes of the set of risk factors. The at least one memory and the computer program code are further configured to, with the at least one processor, cause the apparatus at least to determine an expected effect of an intervention program on the disease risk. The at least one memory and the computer program code are additionally configured to, with the at least one processor, cause the apparatus at least to conditionally implement the intervention program for the person based on the expected effect of the intervention program.

### BRIEF DESCRIPTION OF THE DRAWINGS

For proper understanding of the invention, reference should be made to the accompanying drawings, wherein:

FIG. 1 is a block diagram representing a generic decision support system for patient data.

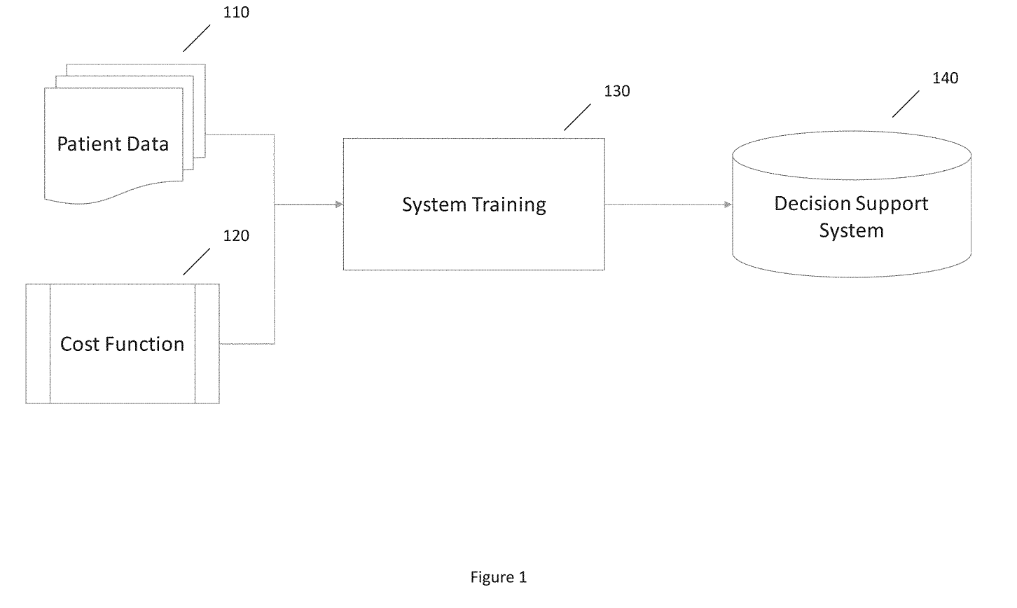

FIG. 2 is a block diagram of a decision support system which tries to apply machine learning without reducing the dimensionality of the input data and hence ends up being suboptimal.

FIG. 3 is a block diagram of a decision support system which extracts a subset of the input data to use in machine learning but ends up being suboptimal due to potentially ignoring useful inputs.

FIG. 4 is a block diagram of a decision support system using purely evidence based predictions and therefore unable to adapt to a custom cost function or risk factors not in the scientific literature or of special interest for a particular cohort.

FIG. 5 is a block diagram of an exemplary evidence based prediction model, consistent with embodiments of the present invention, which takes in risk factors and transforms them into a disease risk based on the scientific literature.

FIG. 6 is a simplified block diagram of an embodiment of the present invention including an Evidence Based Prediction (EBP) model, along with a Combined Optimal Learning System (COLS).

FIG. 7 illustrates a method according to certain embodiments of the present invention.

FIG. 8 illustrates risk estimation with no intervention program, according to certain embodiments of the present invention.

FIG. 9 illustrates risk estimation with the intervention program, according to certain embodiments of the present invention.

FIG. 10 illustrates the aggregation of data over diseases to get financial saving for a single user, according to certain embodiments of the present invention.

FIG. 11 illustrates an example for the analysis of an intervention program at a population level, according to certain embodiments of the present invention.

FIG. 12 illustrates a system according to certain embodiments of the present invention.

### DETAILED DESCRIPTION

There is a need for systems that can process large amounts of patient data to make better decisions and can do so in a way which can be clearly linked to the existing scientific literature. Certain embodiments of the present invention provide methods and systems to help make better automated decisions about patient conditions from data. As will be described below, one aspect of certain embodiments of the present invention is a way to use many dimensions of patient data in a machine learning system effectively. This allows decisions that are better than those provided by existing systems because certain embodiments of the present invention can look at more dimensions of patient data individually and in combination without suffering from the curse of dimensionality.

Conceptually, certain embodiments of the present invention provide a learning system and a decision system. Roughly speaking, the learning system may take some patient information referred to as a training set, perform some analysis on the same, and produce a decision system. Once the decision system is built, new patient data can be fed into the decision system for automated decisions. Splitting a system into a learning system and a decision system can be done for clarity of exposition. In the following, the description of the learning system is the focus.

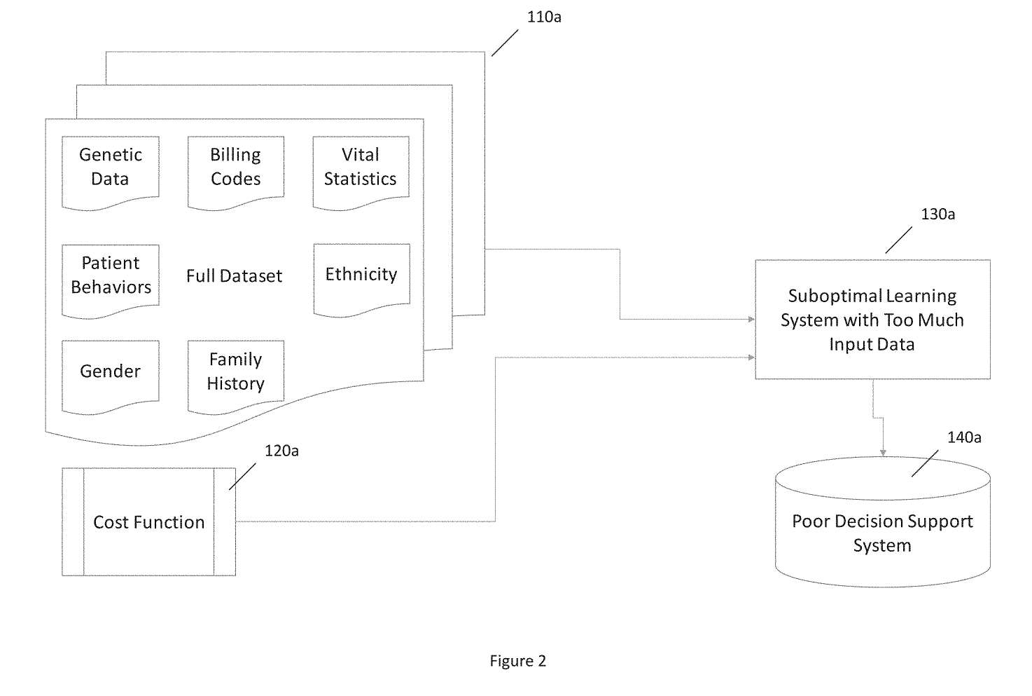

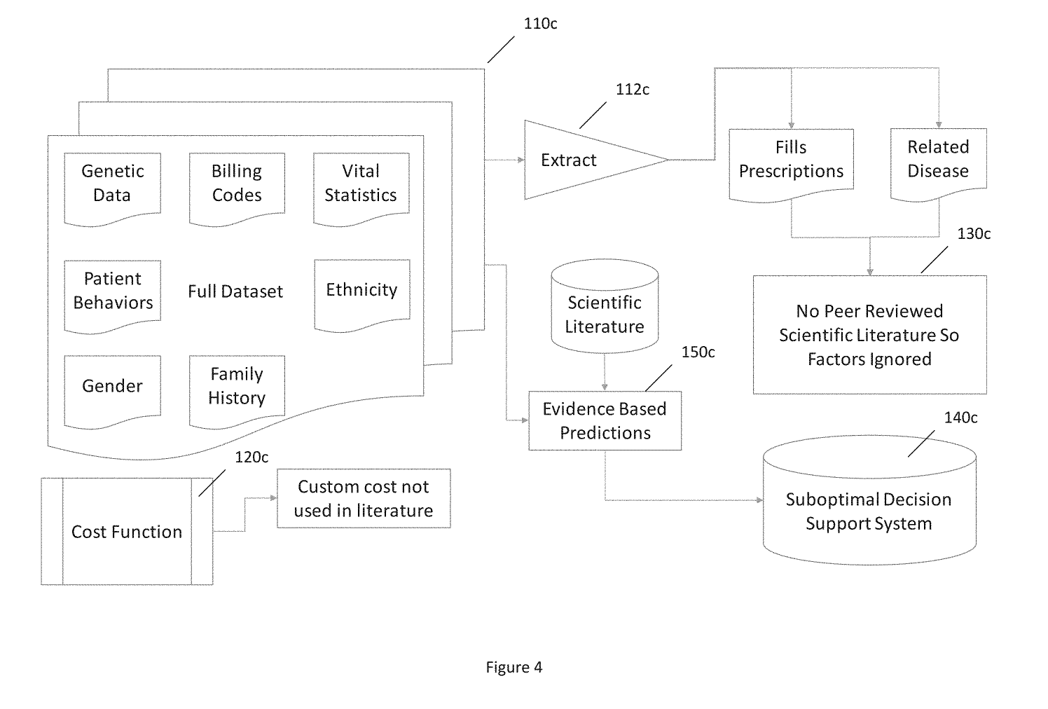

As discussed above, there are many applications where it would be valuable to train a machine learning system with a custom (usually asymmetric) cost function to make a decision using a large amount of patient data. FIGS. 2, 3, and 4 illustrate various difficulties in using all available data. With certain embodiments of the present invention, a larger set of input data can be used than in previous machine learning systems (i.e., addressing the curse of dimensionality) while still being able to adapt to custom cost functions and important pieces of data without sufficient analysis in the existing scientific literature.

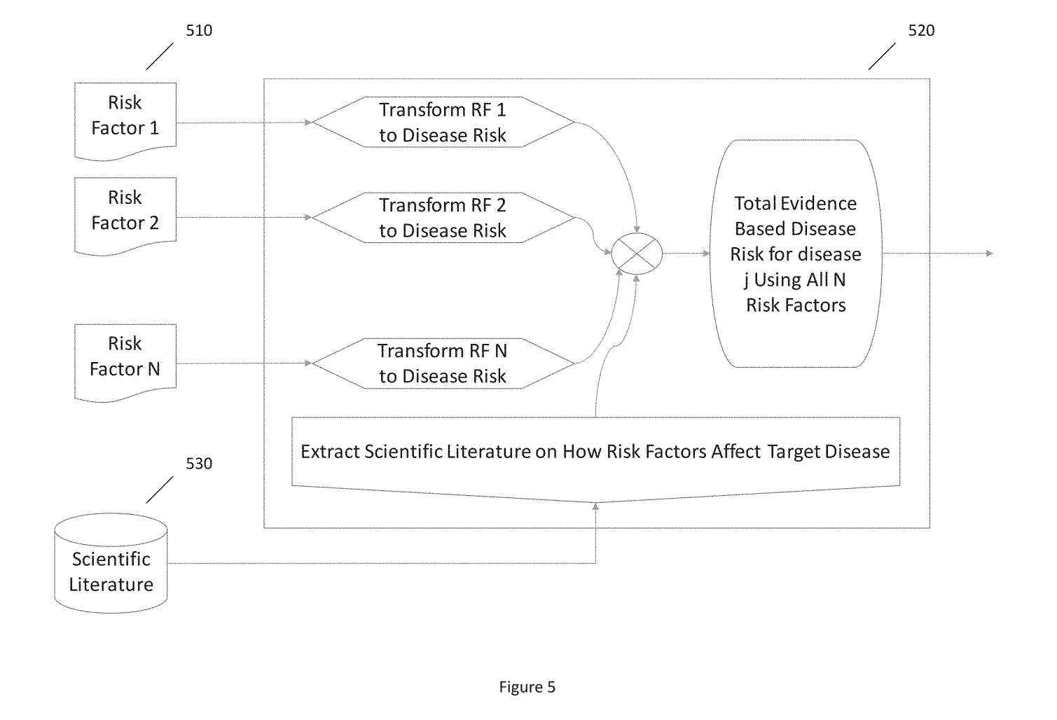

Certain embodiments of the present invention may work by building a multi-stage (for example, two-stage) machine learning system. The first stage may involve “Evidence Based Predictions” or EBP, as shown in FIG. 5, which may take as input patient data and may map that to predicted disease risks. At a high-level, the EBP may work as follows. For each possible risk factor Rᵢ and disease of interest Dⱼ, there may be k previously identified scientific publications Eᵢⱼₖ which specify the risk of developing the disease Dⱼ as a function of the risk factor Rᵢ. How the publications Eᵢⱼₖ may be found is set forth, for example, in U.S. Prov. Pat. Appl. No. 62/440,018, filed Dec. 29, 2016, the entirety of which is incorporated herein by reference.

The disease risk Dⱼ may be computed as a function of the relevant risk factors ƒⱼ(R₁, R₂, . . . , R_{N}). For example, one might identify a log odds ratio for developing disease Dⱼ based on Rᵢ as a function of paper Eᵢⱼₖ.

Through a slight variation, the log odds for the partial risk of developing disease Dⱼ based on risk factor Rᵢ as determined by publication k can be denoted as Eᵢⱼₖ(Rᵢ). The total risk can be determined as Dⱼ=Bⱼ exp[ΣᵢΣₖ(WᵢⱼₖEᵢⱼₖ)] where the coefficient Wᵢⱼₖ may depend on the quality/accuracy of publication k. The coefficient Bⱼ may be a normalization constant designed so that the predicted disease incidence for disease j matches the expected incidence in the population of interest. As a result, the EBP may map a set of patient data which we call risk factors R₁, . . . , R_{N} into M disease risks Dⱼ according to the previous formula.

One aspect of the EBP may be that the complexity of building the EBP may be linear in the number of risk factor—disease combinations. That is if there are N risk factors and M diseases, then the complexity of the EBP may be N×M because one may find a set of scientific papers for each risk factor and disease. In essentially every known general machine learning algorithm, the complexity of handling N×M input dimensions is super-linear (and often exponential) in the dimension N×M.

FIG. 5 is a block diagram of an exemplary evidence based prediction model, consistent with embodiments of the present invention, which takes in risk factors and transforms them into a disease risk based on the scientific literature. As shown in FIG. 5, risk factors 510, designated as 1 through N, can be processed in the EBP 520. The EBF 520 can transform each risk factor into a disease risk and extract scientific literature from scientific literature database 530. Based on the disease risks and scientific literature, the EBF 520 can provide a total evidence-based disease risk for disease j using all N risk factors.

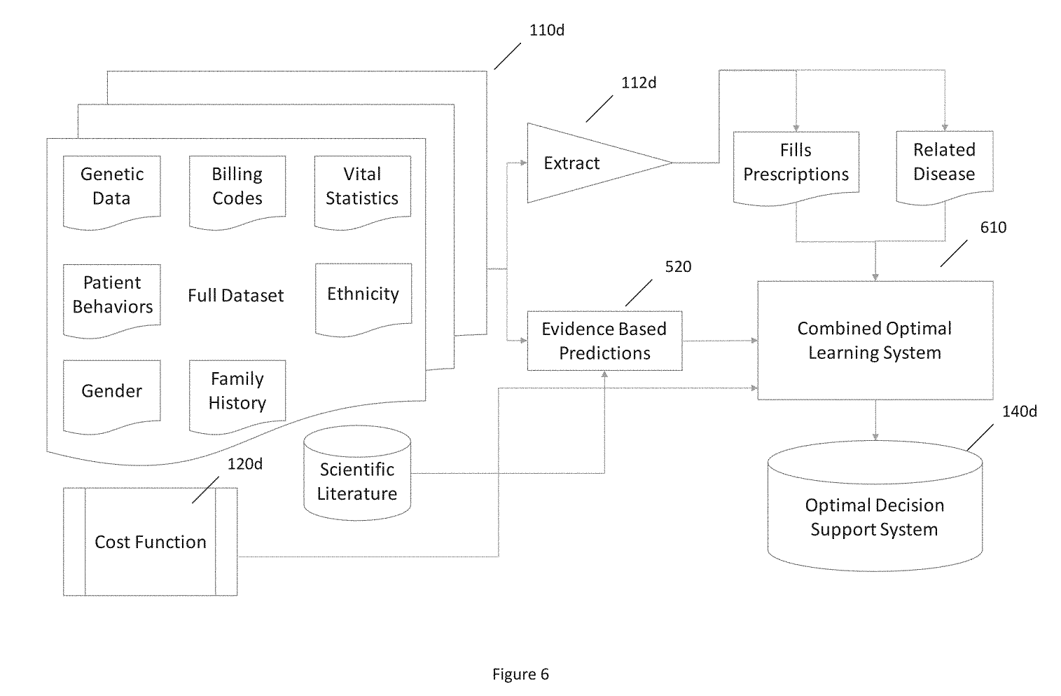

A second stage may include a machine learning system which may use whatever input factors are available along with the disease risks from the EBP system and the desired cost function. An embodiment of the present invention is shown in FIG. 6 which may use the EBP in conjunction with an additional machine learning stage we refer to as the Combined Optimal Learning System (COLS).

FIG. 6 is a simplified block diagram of an embodiment of the present invention including an Evidence Based Prediction (EBP) model, as illustrated in FIG. 5, along with a Combined Optimal Learning System (COLS) to take the output of the EBP as well as additional risk factors and produce an optimal decision support system.

As shown in FIG. 6, patient data 110d is extracted at 112d from a full dataset, to provide fills prescription and related disease data. This fills prescription and related disease data, together with a cost function 120d, can serve as inputs to a combined optimal learning system 610. The combined optimal learning system 610 may also receive evidence based predictions from EBP 520, which may make its assessment based on the patient data 110d and scientific literature. The result can be a decision support system 140d that is an optimal decision support system.

There may be many ways to build the EBP and COLS. It should be understood that part of the power of certain embodiments of the present invention is splitting the intractable probability of learning from high dimensional data into two or more interacting stages, in this case the EBP and the COLS, in order to simplify the problems described herein.

For example, imagine one were trying to learn to diagnose which patients in a particular cohort were at high risk of having a stroke. Furthermore, imagine that one considered a wide range of potential risk factors including things like a patient's status for age, gender, blood pressure, prevalence of chronic pain, hypertension, diabetes, hyperlipidemia, hepatitis, and so on. Finally, imagine that the cost was asymmetric so that the cost of a missed detection (incorrectly diagnosing a patient as not at risk of stroke) is much more costly than a false alarm (incorrectly diagnosing a possible stroke when none is present or would occur).

One may first feed one or more of or all of the risk factors into the EBP to obtain the EBP stroke risk Dⱼ (R₁, . . . , R_{N}) as described previously. One may then take risk factors which the EBP may not provide much weight on either way or which one may want to train further and feed these into the second stage machine learning system along with Dⱼ. For example, imagine that a hospital records data on patients who suffer from cardiac arrhythmias and hypertension and refer to this as risk factors R₁ and R₂. In the second stage which we refer to as COLS, one could train a support vector machine (or other machine learning techniques) with inputs Dⱼ (R₁, . . . , R_{N}), R₁, and R₂ using any desired asymmetric cost.

Since Dⱼ(R₁, . . . , R_{N}) may capture the scientific consensus of how a large array of risk factors may affect stroke, the system may use all of this information (or subset of this information) as encapsulated in Dⱼ. Since we may be further interested in risk factors R₁ and R₂ for this particular cohort, a support vector machine system could learn and adapt on how to further use R₁, R₂ with an asymmetric cost.

The end result may be a system that efficiently handles a high dimension of risk factors known to be related to stroke while also being able to efficiently adapt to a particular cohort where the specific risk factors R₁ and R₂ are potentially relevant.

As summarized previously, certain embodiments of the present invention provide ways to build automated decision support systems which can efficiently handle high-dimensional patient data by using a multi-stage (for example, two-stage) machine learning system, illustrated by way of example in FIG. 6.

The Evidence Based Prediction (EBP) stage may include a system which may take as input N risk factors for a patient, R₁, . . . , R_{N}, and produce M disease risks D₁, . . . , D_{M}. An exemplary embodiment of the EBP is the Genetic and Environmental Risk Engine (GERE). For a detailed description of the GERE, we incorporate herein by reference U.S. patent application Ser. No. 14/104,861, filed Dec. 12, 2013.

The following is an example of building the COLS stage. While the particular example provides an illustration, the system is not limited to the particular example set forth herein and can therefore be applied to a wide array of other problems and data.

The example set forth here is predicting whether a patient has had a stroke. This could be useful information for fraud detection, billing analysis or a wide variety of other scenarios. The same or a similar approach could be used to predict if a patient may have a high risk of having a stroke in the future (for example, by training with different target data). We use detecting a past stroke as an example since that data is more readily available for us and also for others to reproduce the results of this example.

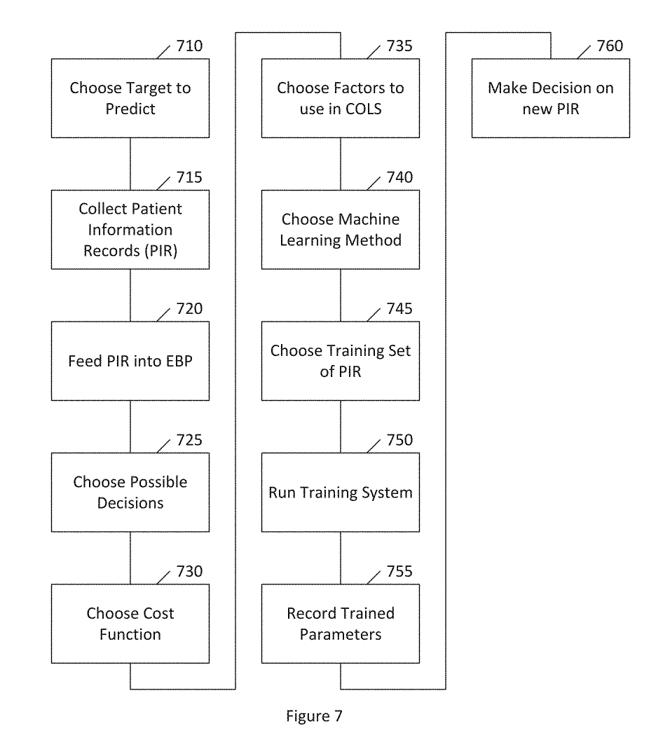

FIG. 7 illustrates a method according to certain embodiments of the present invention. As shown in FIG. 7, a method can include, at 710, choosing a target to predict. In this example, the system is trying to predict whether a patient has had a stroke, as recorded in his or her hospital record. Hence, the system can use a set of hospital records for a group of patients for training and evaluation.

At 715, the method can include collecting the patient information records (PIR) which may serve as input data. Each PIR may be a list of N numbers indicating information about a patient. These items of information can be referred to as “risk factors” R₁, R₂, . . . , R_{N}. For example, these could include the following variables: history of cardiac arrhythmias (i.e., whether the patient has had a recorded event of a cardiac arrhythmia in the past or not), age, alcohol consumption, body mass index, ethnicity, smoking, gender, past cardio-vascular disease, physical activity level, diabetes, hypertension, depression, dementia, chronic pain, chronic kidney disease, hyperlipidemia, hepatitis or any other desired variable.

At 720, the method can include feeding each PIR into the EBP to obtain the disease risks D₁, D₂, . . . , D_{M}. In the current example, D₁ would correspond to the EBP predicted risk for stroke.

At 725, the method can include choosing the possible decisions. In this example, the decisions can be “0” corresponding to “no predicted stroke” and “1” corresponding to “predicted stroke”.

At 730, the method can include choosing a cost function. In this example, there could be a cost of 7 for deciding “0” when a stroke is present and a cost of 1 for deciding “1” when no stroke is present. The cost for a correct decision may be 0.

At 735, the method can include choosing the factors to use in the COLS. For simplifying this example, R₁ (history of cardiac arrhythmia), R₂ (prevalence of hypertension) and D₁ (EBP prediction for stroke) can be taken as the inputs to the COLS.

At 740, the method can include choosing a machine learning method to train the COLS. In this example a logistic regression is used, although many other choices are possible as well. Again, for simplifying this example, the logistic regression implemented in the python scikit-learn software package or any other desired software package can be used.

At 745, the method can include choosing a training set of PIRs along with associated outcomes for the target variable. In this example, patient information records can be used from a major health care organization in Phoenix, Ariz.

At 750, the method can include running the training system to minimize the desired cost of the decision on the training set. In this example, sciki-learn can be used to find a logistic regression function taking in R₁, R₂ and D₁ as inputs and making stroke predictions to minimize our asymmetric cost function.

At 755, the method can include recording the trained parameters. This may be the end of the training step and the full prediction system may now be used on new patient information records (PIRs) not previously seen. In this example, one may let L(R₁, R₂, D₁) be the decision trained logistic regression function that has been trained. Thus the full decision may be L(R₁, R₂, D₁). Note that since D₁ depends on all the risk factors, the decision function could also be written as L(R₁, R₂, D₁(R₁, R₂, . . . , R_{N})) to more fully illustrate the two-stage structure and its dependence on the full data available for each patient.

At 760, the method include making a decision on a new patient information record. When a decision is desired on a new PIR, the system can evaluate the EBP and feed the EBP prediction along with the additional factors into the COLS function that has been trained. In our example, this may mean feeding R₁, R₂, . . . , R_{N} into the EBP to obtain D₁ and then feeding R₁, R₂, and D₁ into the logistic regression to obtain the decision L(R₁, R₂, D₁(R₁, R₂, . . . , R_{N})).

When a logistic regression was run purely on the cardiac arrhythmia and hypertension data (R₁, R₂), the average cost on the training set was 0.1978 while the average cost on a test set which had not been used for training was 0.2245. It is a practice in machine learning to train a system with samples of “training data” and estimate the “out-of-sample” cost by testing on a new set of data which was not used in training.

When only the EBP of the GERE was used, there was an average test set cost of 0.1932. That is, the cost using the EBP was lower than with logistic regression using only R₁ and R₂. This is because the EBP benefits from the effort of the scientific literature in understanding stroke prediction and uses a larger number of risk factors than a logistic regression with only R₁ and R₂.

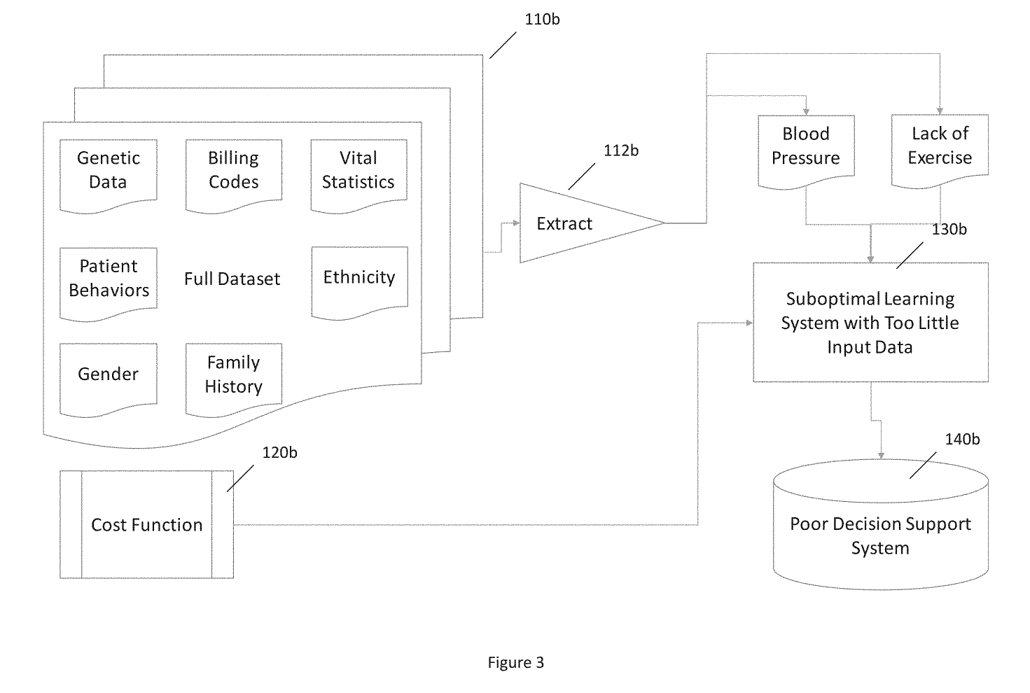

Effectively, the logistic regression using only R₁ and R₂ is an example of the suboptimal system shown in FIG. 3 which uses too little input data. Similarly, the EBP is an example of a system such as the one in FIG. 5 which can use more input risk factors and hence do better.

The average error was also evaluated in a logistic regression trained with a set of available data including cardiac arrhythmia, age, alcohol use, blood pressure, body mass index, ethnicity, smoker, gender, past cardio-vascular disease, physical activity level, diabetic, hypertension, depression, dementia, chronic pain, chronic kidney disease, hyperlipidemia, and hepatitis, as an example of the suboptimal system in FIG. 2. Doing so produced an average test set cost of 0.2259.

This is worse than both the EBP and also the simple two-factor logistic regression because of the curse of dimensionality: traditional machine learning with too many factors quickly becomes infeasible. While the training error using a logistic regression on R₁, R₂, . . . , R_{N} may be indeed relatively low at 0.1856, this is because the logistic regression may be overfitting artifacts in the data. The overly complex model performs poorly on the test set because it does not generalize well.

Certain embodiments of the present invention, however, may combine the output of the EBP along with R₁ and R₂ to obtain an average test set error of 0.1807. This is just one example of how certain embodiments of the present invention may provide improvements over existing systems. By using an EBP which can incorporate a large amount of inputs using predictions from the scientific literature and a second stage machine learning algorithm which can adapt to data which the scientific literature does not consider in detail (but which are apparently relevant for this cohort), certain embodiments of the present invention may obtain better performance than either the EBP in FIG. 5, the suboptimal system with only a few factors in FIG. 3, or the suboptimal system with too many inputs in FIG. 2.

Certain embodiments of the present invention may have various benefits and/or advantages. One quantitative advantage is that our multi-stage (for example, two-stage) machine learning approach can handle high-dimensional data better than other machine learning systems. As illustrated in the stroke prediction example, certain embodiments of the present invention have been reduced to practice and tested on real data to show that they can outperform other systems.

In addition to this quantitative advantage, the exemplary two-stage system has a qualitative advantage. By using the EBP to accurately summarize a wide range of patient data into disease risks, certain embodiments of the present invention let a system designer focus on special data that might be available for a given cohort or organization without having to build a full model for all patient data.

This qualitative advantage can be seen from an example. Imagine that a hospital analytics technician is asked to build a system to screen patients to determine if they should receive a suggestion to attend a weight management program to reduce the risk of developing type 2 diabetes. Without the present invention, the technician would have roughly two choices: simply go by the standard medical literature and risk factors to select patients or use a purely statistical approach. In the case where many years of past billing codes are available to inform the decision, purely using the medical literature seems suboptimal. But using a purely statistical machine learning approach to learn everything about the patient's diabetes risk only from the data is usually too hard, as illustrated by the previous example with overfitting.

Certain embodiments of the present invention let the technician use an EBP (such as GERE, as described in incorporated U.S. patent application Ser. No. 14/104,861, filed Dec. 12, 2013) to get a good basic estimate of the diabetes risk based on standard medical risk factors from the scientific literature and combine that with the hospital's own billing code data to build an optimized custom solution.

Thus, certain embodiments of the present invention provide systems and methods for building a decision support system to evaluate the likelihood of a condition in a patient and suggest a decision. A multi-stage (for example, two-stage) machine learning approach may combine an evidence based prediction model with a second machine learning stage in order to handle high-dimensional patient data efficiently. The evidence based prediction model may use information from the scientific literature to map patient risk factors into disease risks. The resulting disease risks can then be combined with arbitrary input factors to train a machine learning system to make optimal decisions.

Additionally, certain embodiment of the present invention may provide a system and method for analyzing the financial and health benefits of a health intervention program.

A health intervention program is a program in which one or multiple health risk factors are targeted for improvement. While intervention programs seem to be promising preventive actions, justifying financial benefits of such programs is not as straightforward, as explained by Cohen J. T., et al. in “Does preventive care save money? Health economics and the presidential candidates,” New England Journal of Medicine, 2008, Mass Medical Soc., the disclosure of which is fully incorporated by reference herein.

The following example helps to illustrate why justifying the financial benefits of intervention programs is not as straightforward. Consider a population of one million initially healthy individuals and 100 health conditions, each with a probability of 0.1%, which may independently develop in the population in the upcoming year. Assume a fixed treatment cost of $5,000 needs to be paid for each incidence of disease in the next year. The incidence data implies there will be an average of

$$100 ( diseases ) \times \frac{0.1}{100} ( Icidence ) \times 1 , 000 , 000 ( Individuals ) = 100 , 000$$

new cases of disease next year in the population, which results in a treatment cost of 100,000×5000=$500M.

Now consider a health intervention program that costs $600 per individual. Assume that after applying the intervention program the risk of getting the diseases completely vanishes. Under this assumption, with the intervention program the treatment cost of the disease will go to zero; but instead one needs to pay an intervention program cost of 600×1,000,000=$600M. This cost is even larger than the required treatment cost calculated in the absence of the intervention program.

Now assume there is a system and method that helps select the 50% of the population who are at highest risk of developing any of the 100 diseases. The risk in the rest of the population is negligibly small. With this assumption, the same outcome can be obtained while applying the intervention program to only half of the population. The needed prevention program cost will be 50/100×1000,000 (Individuals)×600 (Intervention cost)=$300M. In this case, running the intervention program results in $200M in savings, which is 40% of the required treatment cost in the absence of any intervention program.

As demonstrated in the above examples, there is a fine interplay between the parameters that contribute to the financial outcomes of an intervention program. This highlights a need for an accurate risk analysis before the health management program is performed.

The illustrative example mentioned above was simplified in multiple dimensions. First, it was assumed that the risk of disease completely vanishes as a result of the intervention program. In practice, the disease risk will always remain greater than zero. To run the intervention program(s), one may need to identify those individuals who will benefit the most from the intervention program(s). Second, it was assumed the incidence rate and treatment cost of all diseases are similar. In practice, diseases may have different incidence rates and costs. Third, it was assumed that all individuals in the population are initially healthy. In practice, at any time there may be a number of pre-existing diseases in the population. These considerations may all be accounted for when performing risk analysis. These details further highlight the need for an accurate and comprehensive analysis of the intervention programs.

Healthcare costs come from diseases and disease incidences may be governed by disease risk factors (such as BMI, blood pressure, lipid panel data and smoking status). This implies a proper risk analysis may be based on the analysis of risk factors. There is a need for a system and method that evaluates an intervention program based on the risk factor data and models the effect of this intervention program simultaneously on a large number of diseases.

Certain embodiments of the present invention provide such a system and method. The resulting system and method may be based on an evidence-based disease risk prediction engine (EBPE) as described above.

A point of strength of the presented system and method may be its ability to perform the risk analysis even if part of the data for the users is missing. This may be an important feature of the system as existing healthcare applications are usually missing a portion of the health data.

The inputs that may be used by the method can be categorized into four categories. A first category, which can be called input 1 for ease of reference only, can be characteristics of the intervention program. Specifically, this category can include the cost of the program, the risk factors that may be addressed by the program, and the efficacy of the program with respect to each addressed risk factor.

A second category, which can be called input 2 for ease of reference only, can be an individual's risk factor data. This may include information such as the individual's age, gender, ethnicity, BMI and blood pressure. The method can analyze cases where some of the risk factor data is missing. For example, the individual of interest may be a 55 year old Hispanic male with a body mass index (BMI) of 31 kg/m2 and a blood pressure of 155/90 mmHg. It may be known that he is a smoker who smokes 20 cigarettes per day; but data on lipid panel, alcohol intake and other potential risk factors may be missing.

A third category, which can be called input 3 for ease of reference only, can be time horizon of interest. This time horizon may refer to the time period over which the financial/health benefits of the intervention program may be evaluated. Typically, intervention programs are more valuable over longer periods of time.

A fourth category, which can be called input for ease of reference only, can be the set of diseases of interest and their annual treatment cost. If this information is not available, it may be assumed that all diseases are relevant in the analysis, and the annual cost of the diseases may be taken from publicly available literature.

Certain embodiments of the present invention may be designed to analyze the effect of an intervention program on a single individual. However, the analysis can be aggregated over all the individuals in a population (or subset thereof) to evaluate the intervention program at a population level.

The analysis may start by assessing the risk of the individual under study for the set of diseases of interest. The diseases may be analyzed independently from each other.

An exemplary method may utilize the evidence-based prediction approach described above to evaluate the disease risks. An embodiment may use disease risk factor data collected from the literature to perform risk assessment. In that case, the disease risk equation may take the form D=ƒ(exp(Σᵢ₌₁^{N}Σₖ(WᵢₖEᵢₖ))), where D is the estimated disease risk calculated based on N different disease risk factors. The data for each risk factor may be taken from a number of different scientific publications. The variable Eᵢₖ denotes the log effect size reported for the i^{th} risk factor in the k^{th} publication used for this risk factor. The coefficient Wᵢₖ denotes a measure of quality for the publication mentioned above. The function ƒ(.) may be used to map the aggregate effect size calculated over all the risk factors (or subset thereof) to the disease risk.

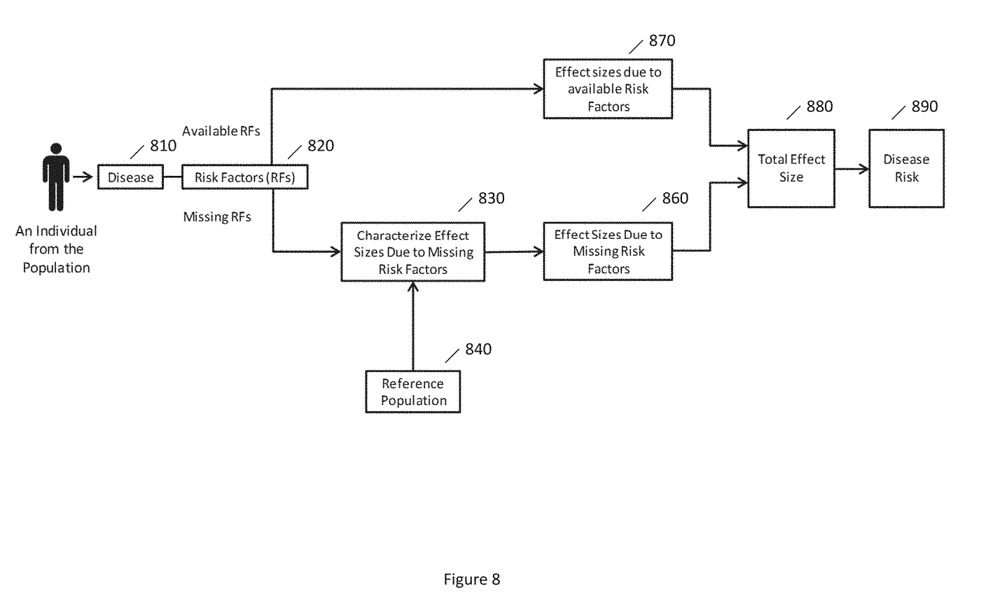

The equation mentioned above can be simplified as D=ƒ(exp(Σᵢ₌₁^{N}Eᵢ)), where Eᵢ=Σₖ(WᵢₖEᵢₖ) is the aggregate log effect size calculated for the i^{th} risk factor. With a slight variation of notation, the equation can be further simplified as D=ƒ(Πᵢ₌₁^{N} Eᵢ), where Eᵢ represents the actual effect size (no longer the log effect size) due to the i^{th} risk factor (FIG. 8).

The disease risk equation mentioned above is based on the evidence-based disease risk prediction approach. However, similar methods apply if the disease predictions are obtained based on a Combined Optimal Learning System (COLS) in which both evidence-based predictions and other inputs from the Patient Information Records (PIR) could be incorporated.

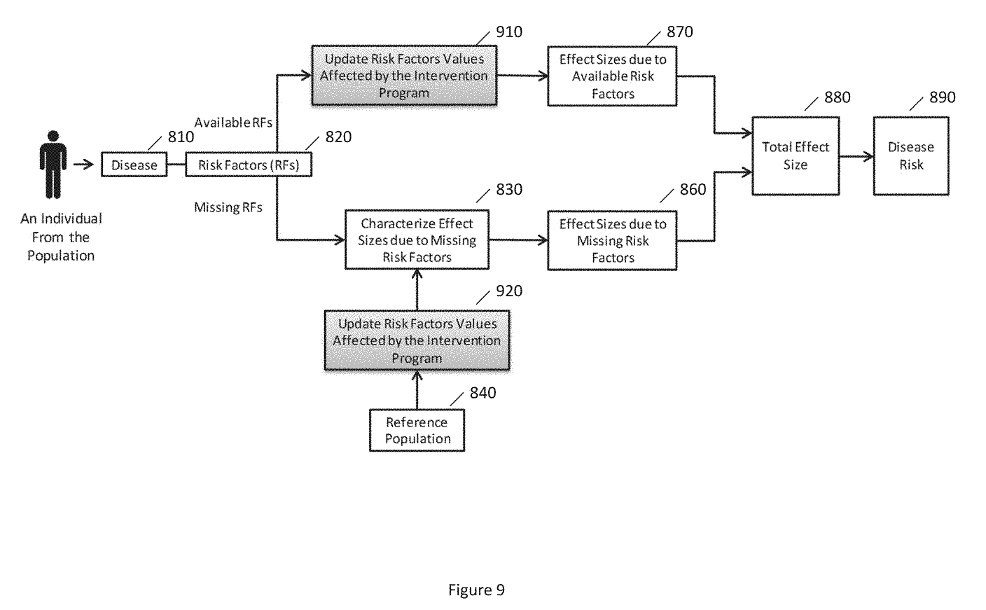

The EBPE may be applied separately to each disease and may be used to estimate the disease risks in the presence and absence of the intervention program (FIGS. 8 and 9).

An intervention program may aim to improve the value of one or more risk factors. To account for the effect of the intervention program, the effect sizes of the addressed risk factors may be modified to match their updated values. The updated values of the risk factors may be, in turn, determined by the efficacy of the selected intervention program (FIG. 9).

An exemplary method is illustrated through an example. Consider a disease with only four risk factors R₁, R₂, R₃ and R₄. Let the values of the risk factors before applying the intervention program be V₁, V₂, V₃ and V₄, respectively. Also, let E₁, E₂, E₃ and E₄ be the effect sizes corresponding to the values V₁, V₂, V₃ and V₄. With these assumptions, the resulting total effect size for the individual will be E=E₁×E₂×E₃×E₄, which should be used as the basis for calculating the underlying disease risk.

Next, consider an intervention program that may be aimed to address risk factors R₂ and R₃. Let the updated values of these risk factors with the intervention program be V′₂ and V′₃, respectively, corresponding to the effect sizes E′₂ and E′₃. The updated total effect size with the intervention program will be E=E₁×E′₂×E′₃×E₄, which may be used as the basis for calculating the disease risk in the presence of the intervention program.

The EBPE may have the ability to perform a risk analysis even if only a subset of risk factor data is available for the user of interest. To explain how this is achieved, two cases can be considered: (Case 1) handling missing risk factors in the absence of the intervention program; and (Case 2) handling missing risk factors in the presence of the intervention program.

Case 1 is the handling of missing risk factors in the absence of the intervention program. Assume that among the N risk factors for a certain disease, data for Nₐ risk factors is available and data for Nₘ risk factors is missing. The effect size term can be broken into two parts: Πᵢ₌₁^{N}Eᵢ=(Πᵢ₌₁^{N}^{ₐ}Eᵢ)(Πᵢ₌₁^{N}^{ₘ}Eᵢ), where the first term corresponds to the risk factors with available data and the second term corresponds to the risk factors with missing data. For the risk factors with available data, the corresponding effect size Eᵢ can be directly used. For the risk factors with missing data, a reference population can be used as a training data set in which all the risk factor data (or subset thereof) may be available to estimate the multiplication of the effect sizes due to the missing risk factors (that is, the second term in the equation above). Specifically, there may be a need for a statistical model—developed based on the reference population—in which the input parameters may be the known risk factors and the response variable may be the multiplication of effect sizes due to the missing risk factors.

The final effect size for each individual may be calculated by multiplying the effect sizes for the available risk factors with the estimated multiplication of effect sizes from the missing risk factors obtained from the statistical model (FIG. 8).

Case 2 is the handling of missing risk factors in the presence of the intervention program. In this case, once again a statistical model may need to be developed based on the reference population. The difference is that before developing such a model, the effect sizes of the risk factors that are addressed by the intervention program may be updated in the reference population. This implies the estimation may be performed after accounting for the effect of the intervention program (FIG. 9).

In the above analysis, the output of the statistical model may be the multiplication of the effect sizes due to missing risk factors, which may vary depending on whether the intervention program is applied or not. However, the input to the statistical model may be the values of known risk factors before the intervention program is applied.

Consider the example mentioned in paragraph [00101]. Assume that the data for the two risk factors R₁ and R₂ are available (V₁ and V₂, respectively, as mentioned before) but the data for the two risk factors R₃ and R₄ is no longer available. Now we can consider the following two cases: a case when no intervention is applied, and a case when an intervention program is applied.

In the case when no intervention is applied, the effect sizes due to R₁ and R₂ are easily obtained. (They are E₁ and E₂ respectively, as before.) It remains to estimate the multiplication of E₃ with E₄ for the user. To this end, a reference population may be used in which the values of the four risk factors R₁, R₂, R₃ and R₄ are available. A statistical model may be trained based on this reference data in which the input variables are the two risk factors R₁ and R₂, and the (univariate) response variable is the multiplication of E₃ with E₄. The model trained on the reference population may then be used to estimate the multiplication of E₃ with E₄ for the profile of interest, that is, R₁=V₁ and R₂=V₂.

An example of the statistical model that can be used for this purpose is the following. Identify all individuals in the reference population that match the profile of the user of interest. That is, individuals for which R₁=V₁ and R₂=V₂. (In practice, some level of discrepancy is acceptable.) Call this set S. Calculate the average of E₃×E₄ across all individuals in the set S to estimate E₃×E₄ for the user of interest.

In a second case, a situation in which the intervention program is applied can be considered. An intervention program, in this example, can address the two risk factors R₂ and R₃. The goal may be to estimate the overall effect size in the presence of the intervention program for the individual. For the two known risk factors R₁ and R₂, the effect sizes at the presence of the intervention program are E₁ and E′₂, respectively, as before. To estimate the overall effect size for the user, it remains to estimate the multiplication of effect sizes due to the missing risk factors in the presence of the intervention program. To this end, the effect sizes in the presence of the intervention program may be calculated for all individuals in the reference population. Then a statistical model may be developed in which the input variables are the two risk factors R₁ and R₂, and the response variable is the multiplication of effect sizes due to the two missing risk factors R₃ and R₄ in the presence of the intervention program. Because only R₃ is addressed by the intervention program, only the effect sizes for this risk factor will be different compared to the previous case where no intervention program was applied.

A statistical model can be developed as following. For all individuals in the set S (as discussed above) calculate E′₃×E₄ and return the average.

The methods mentioned above for handling the missing data may be based on the estimation of the multiplication of effect sizes due to missing risk factors. Alternatively, it is also possible to estimate the effect size due to each of the individual missing risk factors and multiply the results together to estimate the aggregate multiplication of effect sizes. An advantage with this method is that it may allow one to evaluate the effect size due to each of the individual missing risk factors. A disadvantage, however, is that it indirectly estimates the multiplication of effect sizes due to the missing risk factors and the resulting measure may be a biased estimate of the quantity of interest if the underlying risk factors are correlated.

FIG. 8 illustrates risk estimation with no intervention program, according to certain embodiments of the present invention. For those risk factors with missing data, the effect sizes may be estimated using a reference population. As shown in FIG. 8, an individual from the population may be evaluated for the potential for a specific disease 810. A variety of risk factors (RFs) 820 can be considered. This risk factors 820 can include available RFs and missing RFs.

For missing risk factors, a reference population 840 may be used to characterize, at 830, the effect sizes due to those missing risk factors. This can lead to a determination, at 860, of effect sizes due to the missing risk factors. Meanwhile, the system can also determine, at 870, effect sizes due to available risk factors.

At 880, the effect sizes from 860 and 870 can be combined to form a total effect size. The total effect size can then be used to determine, at 890, a disease risk for the disease specified at 810.

FIG. 9 illustrates risk estimation with the intervention program, according to certain embodiments of the present invention. In general, risk estimation may be as shown in FIG. 8, with some additions. For example, at 910, the risk factor values for available risk factors may be updated as affected by the intervention program. Similarly, at 920, the risk factor values for missing risk factors may be updated as affected by the intervention program.

Thus, the effect sizes of the risk factors that are addressed by the intervention program may be updated based on the effect of the intervention program. This may also apply to the individuals in the reference population that may be used to characterize the effect sizes due to missing risk factors.

The approaches mentioned above for handling the missing data aim to first estimate the total effect size from the disease risk factors and then use the outcome to estimate the disease probability. It is also possible to develop statistical models that directly estimate the disease probability using the data in the reference population. To this end, first the evidence-based disease risk prediction engine may be run to get the disease probabilities for all individuals in the reference population. These disease probabilities may then be used to train the required statistical models. More specifically, the output in such models may be the disease probabilities calculated for individuals in the reference population and the input in the model may be the risk factors available for the profile of interest. There are two general ways to develop such models: globally trained models and locally trained models.

In the case of globally trained models, the individuals used in the model do not necessarily need to match the profile of interest. As a result, the model may be trained based on a big part or all of the data in the reference population. A potential disadvantage with this approach is that it may re-estimate the known effect sizes for the available risk factors and thus may add noise to the estimated measures.

In the case of locally trained models, first a subpopulation of the reference population that may closely match the profile of interest may be identified. This may be the same as the set S discussed above. Then the disease probabilities for the individuals in this set may be averaged to estimate the disease probability for the user of interest with missing data. This approach may rely on a large reference population to ensure that the size of set S is large enough for potential input profiles.

In some cases, the efficacy of the preventive programs may be known in a probabilistic way. For example, a weight control intervention program may reduce BMI by 1 unit in 50% of the cases, by 2 units in 30% of the cases and by 3 units in 20% of the cases. In these cases, if the exact probability of each case is known, the disease risk may be calculated for each of the probable cases and then the final risk for the individual may be obtained by calculating the weighted average of the estimated disease risks with the weights coming from the probability of each case. However, if the exact probability of each case is not available one may perform the analysis based on the average of the modified effect size of the risk factor. For example, consider an individual with BMI of 33. If the individual attends the intervention program mentioned above her BMI at the end of the program may be 32 with probability 50%, 31 with probability 30% and 30 with probability 20%. The BMI values of 32, 31 and 30 may correspond to effect sizes of 3X, 2.5X and 2.1X, respectively. In this case, the modified effect size used in the subsequent analysis may be (3X×0.5)+(2.5X×0.3)+(2.1X×0.2)=2.67X.

Consider a case in which the efficacy of the intervention program is provided in a probabilistic way. When analyzing the risk factors and/or disease probabilities for individuals in the reference population, it may be again ideal to account for all probable combinations of risk factor modifications across the population and develop a model based on each possible case and use the weighted average of the predictions performed by each model to get the final estimate. However, it may be a computationally too expensive task in large populations. In those cases, one can perform the analysis based on the average of measure of interest (that may be multiplication of effect sizes due to missing risk factors or disease probability) calculated separately for each individual in the population. A statistical model may then be developed based on the results. In a second level of simplification, one may use the average of modified effect sizes for each risk factor, as suggested in the previous paragraph. The results are again used to estimate the multiplication of effect sizes due to missing risk factors or disease probability for each individual in the population.

The EBPE may be calibrated so it may calculate the risk of developing the diseases over any desired period of time. For simplicity of presentation, we consider a (popular) case in which the EBPE is used to estimate the risk of developing the diseases over a period of one year. However, a similar analysis may apply to other time frames. With this assumption, the final outcome of the above analysis may be the risk of developing each disease of interest over a one-year time period in the presence and absence of the intervention program (FIGS. 8 and 9).

The one-year risk of developing the diseases calculated by the EBPE may be based on the assumption that the individual does not currently have the disease. In cases where no information is available about the presence or absence of the disease, the EBPE may be used to estimate the risk of the individual currently having the disease. Depending on how the EBPE is trained it may have the ability to either predict the risk of developing a disease assuming the individual is disease free or the risk of the disease being currently present. Let p denote the calculated risk of developing the disease if the individual currently does not have the disease. Also, let P denote the calculated probability of the disease being present if no information is available about the presence or absence of the disease.

Depending on the context, p may denote the one-year risk of developing the disease either in the presence or absence of the intervention program. But P corresponds to the present time and may not be relevant in the context of the intervention programs.

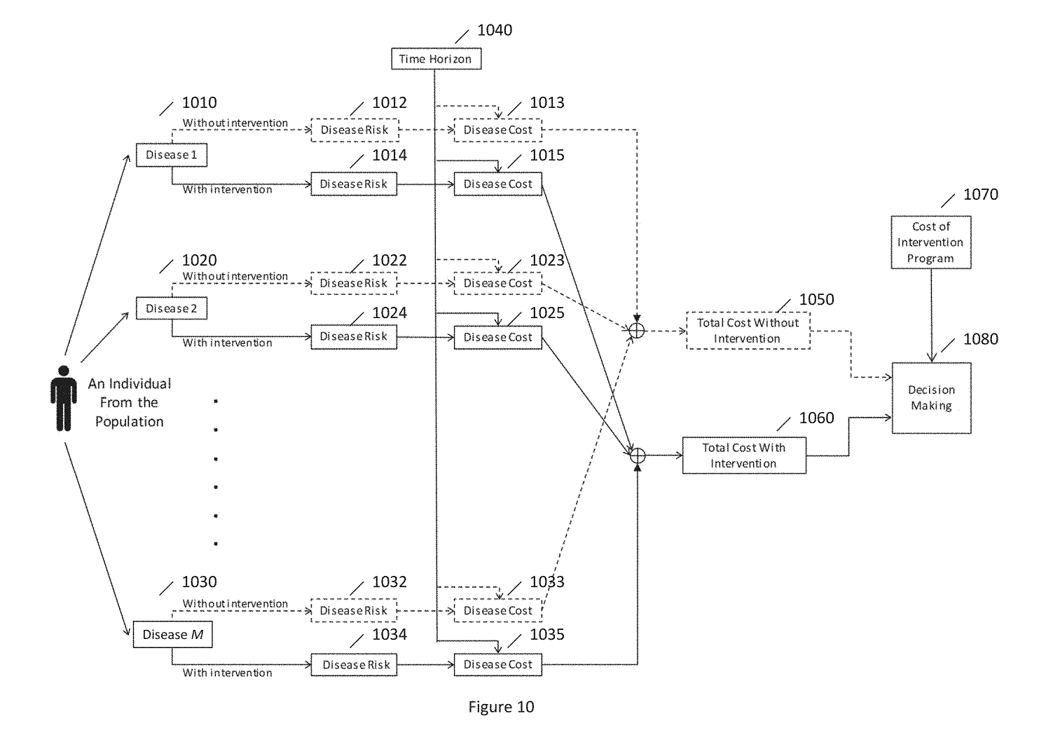

At the next step, the disease risks calculated above may be used to estimate the expected disease cost over the time horizon of interest. As before, the analysis may be performed separately for each disease (FIG. 10). Let T denote the annual treatment cost of the disease of interest. Depending on current status of the individual with respect to the disease, three cases can be studied.

According to a first case, the information about the presence or absence of the disease is available and it indicates that the individual has the disease. In this case, assuming an annual treatment cost of T, the total expected treatment cost over a period of n years, denoted by T₁, is simply given by:

T₁=nT

According to a second case, the information about the presence or absence of the disease is available and it indicates the individual does not have the disease. In this case the one-year risk of developing the disease calculated by the EBPE, p, should be used. The total expected treatment cost in this case, denoted by T₂, is given by:

$${T}_{2} = {\sum }_{i = 1}^{n} ( ( Cost over the year i ) \times Prob [ The disease is present at year i ] ) = {\sum }_{i = 1}^{n} T Prob [ The disease is present at year i ] = {\sum }_{i = 1}^{n} T ( 1 - {( 1 - p )}^{i} ) = T {\sum }_{i = 1}^{n} ( 1 - {( 1 - p )}^{i} )$$

where n is the time span (in years) of the analysis.

According to a third case, no information is available about the presence or absence of the disease. In this case, because no information is provided about the presence or absence of the disease, one may assume the disease is present with probability P and absent with probability (1−P). If the disease is present, the expected treatment cost is given by T₁ calculated as in the first case. On the other hand, if the disease is absent the expected treatment cost is given by T₂ calculated as in the third case. This implies the expected treatment cost in this case, denoted by T₃, is given by:

T₃=PT₁+(1−P)T₂

In some diseases, the treatment cost may be different in each year after the diagnosis of the disease. In these cases, the general approach mentioned above for calculating the expected disease cost remains valid. However, an additional complexity may come from the fact that the time of onset of diseases may be accounted for when performing the cost analysis.

As mentioned earlier, the disease cost analysis may be performed both at the presence and absence of the prevention program. In the above analysis, all the steps remain the same except that the disease development probability p should be determined depending on whether the intervention program is applied or not.

The outcome of the analysis can be the expected cost due to each disease at the presence and absence of the intervention program over the time horizon of interest. The estimated costs calculated for different diseases may then be aggregated to calculate the total expected treatment cost for the individual in the presence and absence of the intervention program (FIG. 10). Let Tᵢₙₜᵥ and T₀ denote the total expected cost at the presence and absence of the intervention program, respectively.

Let Cᵢₙₜᵥ be the cost of the intervention program. The difference between Tᵢₙₜᵥ and T₀ (i.e., T₀−Tᵢₙₜᵥ) should be compared with Cᵢₙₜᵥ to determine whether applying the intervention program results in positive financial gain or not (FIG. 10). Specifically, the expected monetary gain after applying the intervention is T₀−Tᵢₙₜᵥ−Cᵢₙₜᵥ, which could be a positive or negative value. A related measure to determine whether an individual should be assigned to an intervention program or not is return on investment (ROI). Return on investment can be calculated as

$$\frac{{T}_{0} - {T}_{intv} - {C}_{intv}}{{C}_{intv}} .$$

One can set a certain ROI threshold and only assign individuals who pass that threshold to an intervention program.

FIG. 10 illustrates the aggregation of data over diseases to get financial saving for a single user, according to certain embodiments of the present invention. As shown in FIG. 10, an individual from the population can be evaluated with respect to multiple diseases: disease 1 at 1010, disease 2 at 1020 . . . and disease M at 1030. For each of these diseases there can be an evaluation of disease risk without intervention (1012, 1022 . . . 1032) and an evaluation of disease risk with intervention (1014, 1024 . . . 1034). For each of the diseases without intervention, a disease cost (1013, 1023 . . . 1033) can be obtained. Similarly, for each of the diseases with intervention, a disease cost (1015, 1025 . . . 1035) can be obtained. These disease costs can be determined with respect to a time horizon 1040. The disease costs without intervention can be summed to obtain a total cost with intervention 1050, while the disease costs with intervention can be summed to obtain a total cost without intervention 1060. The cost of an intervention program can be calculated or otherwise identified at 1070. Decision making at 1080 can involve determining the difference between the total cost without intervention 1050 and the total cost with intervention 1060. The decision making at 1080 can further involve comparing that difference in total costs with the cost of the intervention program 1070 and analyzing the underlying ROI.

The above analysis provides insights into the financial benefits of the intervention program. To evaluate the health benefits of the intervention program, the disease risks calculated in the process may be used. The fact that the risks may be calculated at a disease/individual level may provide a helpful tool to analyze the health benefits of the intervention program.

The system and method of certain embodiments of the present invention may be designed to analyze the effect of an intervention program for a single individual. Nevertheless, aggregating the individual-level data across a population may provide a versatile tool to analyze the intervention programs in a population. Also, the method can be used to evaluate the effect of multiple intervention programs and perform a comparative analysis among them. Two examples follow.

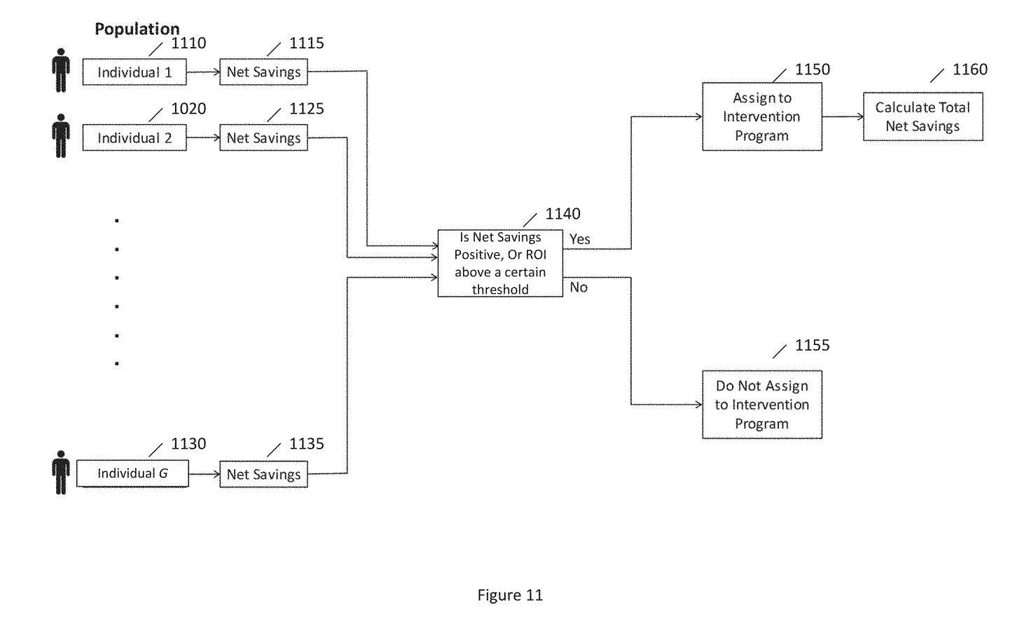

Example 1 is an evaluation of a given intervention program across a population. Consider a case in which the goal is to evaluate the effect of a given intervention across a population. First, an individual-level analysis may be performed to evaluate the effect of the program on each individual in the population (or subset thereof). Next, the results from such evaluation may be used to identify individuals for whom applying the intervention program may result in positive financial revenue, or results in ROI above a certain threshold. Finally, the individuals identified may be assigned to an intervention program (FIG. 11). The risks calculated by the method may be used to determine how the expected number of new cases of each disease in the next year is reduced by the intervention program.

FIG. 11 illustrates an example for the analysis of an intervention program at a population level, according to certain embodiments of the present invention. As shown in FIG. 11, the effect of the intervention program may be evaluated in a population of G individuals: individual 1 1110, individual 2 1120 . . . individual G 1130. For each of the individuals, a respective net savings 1115, 1125 . . . 1135 can be calculated. The program may be evaluated separately for each individual, to determine at 1140 whether a net savings is possible. Those with a predicted positive net saving, or those with ROI above a certain threshold, may be selected for the intervention program at 1150, while those a neutral or negative net savings may not be assigned to the intervention program at 1155. The total net saving can be calculated at 1160 as the sum of net savings of those who are selected for the intervention program.

Example 2 is identifying the best intervention for each individual in the population. Consider a clinic with available infrastructure to intervene with five risk factors: BMI, blood pressure, cholesterol panel, smoking and excessive alcohol intake. The cost and efficacy of the intervention programs may be known. The intervention programs can also be combined to address, multiple risk factors at the same time. In this case the total cost of the intervention program will be the sum of the cost due to the individual intervention programs. The proposed system and method may help the clinic to determine which intervention program or combinations of intervention programs are optimal for each individual given the cost and impact of the intervention programs.

Thus, according to certain embodiments of the present invention a system and method can evaluate the financial and health benefits of a health management intervention program. The analysis may be based on the collective effect of the intervention program on multiple diseases and may be supported by an evidence-based disease risk assessment engine that may be developed based on disease risk factors data collected from the peer reviewed scientific literature. The method can handle users with incomplete health data, a situation that may frequently happen in healthcare applications. The method may be designed to work at an individual level. By aggregating results across individuals in a population, the proposed method can be used to analyze the intervention program in a population in a fine-grained manner. Estimates on the reduced number of new cases of each disease of interest and estimates on the return on investment (ROI) obtained with the intervention programs are representative outputs that may be available through the proposed system and method.

Certain embodiments of the present invention relate to a method for building a system to evaluate the financial and health benefits of a health management intervention program. The method can involve receiving the following information as input: cost of the intervention program to analyze; risk factors that are addressed by the intervention program to analyze; efficacy of the intervention program to analyze; analysis of the effect of the intervention program in an individual and/or a population; risk factor data for the individual/population to be analyzed; and analysis based on the time horizon of interest. The method can also optionally include the following information data as input: past claims data for the individual/population to be analyzed; a set of diseases of interest and their annual treatment cost; and data on the pre-existing diseases in the individual/population to be analyzed.

The method can further include applying an evidence-based prediction engine based on peer reviewed scientific literature, as described above.

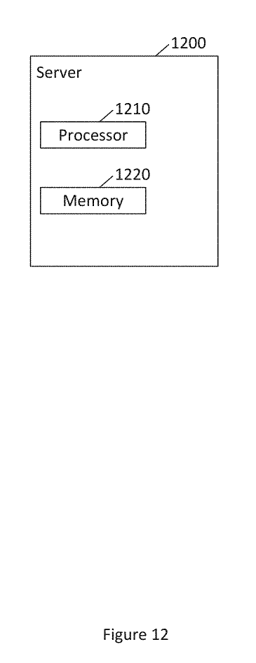

FIG. 12 illustrates a system according to certain embodiments. The system may be a server 1200. The server 1200 may include at least one processor 1210 and at least one memory 1220, including computer program code or other computer program instructions. The processor 1210 may be embodied by any computational or data processing device, such as a central processing unit (CPU), digital signal processor (DSP), application specific integrated circuit (ASIC), programmable logic devices (PLDs), field programmable gate arrays (FPGAs), digitally enhanced circuits, or comparable device or a combination thereof. The processor 1210 may be implemented as a single controller, or a plurality of controllers or processors. Additionally, the processor 1210 may be implemented as a pool of processors in a local configuration, in a cloud configuration, or in a combination thereof. The term circuitry may refer to one or more electric or electronic circuits. The term processor may refer to circuitry, such as logic circuitry, that responds to and processes instructions that drive a computer.

For firmware or software, the implementation may include modules or units of at least one chip set (e.g., procedures, functions, and so on). Memory 1220 may independently be any suitable storage device, such as a non-transitory computer-readable medium. A hard disk drive (HDD), random access memory (RAM), flash memory, or other suitable memory may be used. The memory 1220 may be combined on a single integrated circuit as the processor 1210, or may be separate therefrom. Furthermore, the computer program instructions may be stored in the memory and which may be processed by the processors can be any suitable form of computer program code, for example, a compiled or interpreted computer program written in any suitable programming language. The memory or data storage entity is typically internal but may also be external or a combination thereof, such as in the case when additional memory capacity is obtained. The memory may be fixed or removable.

The memory 1220 and the computer program instructions may be configured, with the processor 1210 for the particular device, to cause a hardware apparatus such as server 1210, to perform any of the processes described above. Therefore, in certain embodiments, a non-transitory computer-readable medium may be encoded with computer instructions or one or more computer program (such as added or updated software routine, applet or macro) that, when executed in hardware, may perform a process such as one of the processes described herein. Computer programs may be coded by a programming language, which may be a high-level programming language, such as objective-C, C, C++, C#, Java, etc., or a low-level programming language, such as a machine language, or assembler. Alternatively, certain embodiments of the invention may be performed entirely in hardware.

One having ordinary skill in the art will readily understand that the invention as discussed above may be practiced with steps in a different order, and/or with hardware elements in configurations which are different than those which are disclosed. Therefore, although the invention has been described based upon these preferred embodiments, it would be apparent to those of skill in the art that certain modifications, variations, and alternative constructions would be apparent, while remaining within the spirit and scope of the invention.

We claim:

1. A method, comprising:
providing, to a learning system, a subset of data from a full dataset of patient information, wherein the subset is expected to relate to a health condition;
providing to the learning system evidence based predictions as to the health condition based on the full dataset informed by scientific literature;
providing a cost function regarding the health condition to the learning system; and
applying machine learning techniques to combine information from the patient dataset with the evidence based predictions in order to produce a mapping that can take in a patient information record and transform it into a likelihood of the desired health condition.

2. The method of claim 1, wherein the full dataset comprises multiple patient information records.

3. The method of claim 1, wherein the likelihood of the health condition comprises a likelihood that a particular person suffers from a particular disease of interest either in the present time or in the future.

4. The method of claim 1, wherein the evidence based predictions are provided by a genetic and environmental risk engine.

5. The method of claim 1, wherein the learning system comprises at least one machine learning method such as logistic regression, linear regression, support vector machine, deep learning, or neural network.

6. The method of claim 1, further comprising:
determining potential risk or liability in accepting a potential future patient into a hospital for treatment or into an insurance plan for coverage, based on the likelihood of the health condition.

7. A method, comprising:
selecting a set of risk factors for a disease for a person;
determining a total effect size and disease risk for the disease based on effect sizes of the set of risk factors;
determining an expected effect of an intervention program on the disease risk; and
conditionally implementing the intervention program for the person based on the expected effect of the intervention program.

8. The method of claim 7, wherein the conditional implementation is further based on the cost of intervention program compared with cost of treatment of the disease multiplied by the likelihood of incurring the disease.

9. The method of claim 7, wherein the determination of the effect size and disease risk and the determination of the expected effect of the intervention program are tied to a time horizon of interest.

10. The method of claim 7, wherein the determination of the effect size and disease risk are based on directly determining effect sizes of risk factors where relevant information is available about the person.

11. The method of claim 7, wherein the determination of the effect size and disease risk are based on determining effect sizes of risk factors based on a reference population, where relevant information is unavailable about the person.

12. An apparatus, comprising:
at least one processor; and
at least one memory including computer program code,
wherein the at least one memory and the computer program code are configured to, with the at least one processor, cause the apparatus at least to
provide, to a learning system, a subset of data from a full dataset of patient information, wherein the subset is expected to relate to a health condition;
provide to the learning system evidence based predictions as to the health condition based on the full dataset informed by scientific literature;
provide a cost function regarding the health condition to the learning system; and
apply the learning system to the provided subset, the evidence-based predictions, and the cost function, to provide a likelihood of the health condition.

13. The apparatus of claim 12, wherein the full dataset comprises multiple patient information records.

14. The apparatus of claim 12, wherein the likelihood of the health condition comprises a likelihood that a particular person will suffer from a particular disease of interest.

15. The apparatus of claim 12, wherein the evidence based predictions are provided by a genetic and environmental risk engine.

16. The apparatus of claim 12, wherein the learning system comprises at least one machine learning method such as logistic regression, linear regression, support vector machine, deep learning, or neural network.

17. The apparatus of claim 12, wherein the at least one memory and the computer program code are further configured to, with the at least one processor, cause the apparatus at least to determine potential risk or liability in accepting a potential future patient into a hospital for treatment or into an insurance plan for coverage, based on the likelihood of the health condition.

18. An apparatus, comprising:
at least one processor; and
at least one memory including computer program code,
wherein the at least one memory and the computer program code are configured to, with the at least one processor, cause the apparatus at least to
select a set of risk factors for a disease for a person;
determine a total effect size and disease risk for the disease based on effect sizes of the set of risk factors;
determine an expected effect of an intervention program on the disease risk; and
conditionally implement the intervention program for the person based on the expected effect of the intervention program.

19. The apparatus of claim 18, wherein the conditional implementation is further based on the cost of intervention program compared with cost of treatment of the disease multiplied by the likelihood of incurring the disease.

20. The apparatus of claim 18, wherein the determination of the effect size and disease risk and the determination of the expected effect of the intervention program are tied to a time horizon of interest.

21. The apparatus of claim 18, wherein the determination of the effect size and disease risk are based on directly determining effect sizes of risk factors where relevant information is available about the person.

22. The apparatus of claim 18, wherein the determination of the effect size and disease risk are based on determining effect sizes of risk factors based on a reference population, where relevant information is unavailable about the person.
